(19) United States
(12) Patent Application Publication
(10) Pub. No.: US 2019/0008920 A1
(43) Pub. Date: Jan. 10, 2019

(54) OPHTHALMIC COMPOSITIONS AND METHODS OF USE

(71) Applicant: Ocugen, Inc., Malvern, PA (US)

(72) Inventors: Rasappa Arumugham, Lansdale, PA (US); Arun K. Upadhyay, West Chester, PA (US)

(73) Assignee: Ocugen, Inc., Malvern, PA (US)

(21) Appl. No.: 16/116,872

(22) Filed: Aug. 29, 2018

Related U.S. Application Data
(63) Continuation-in-part of application No. 15/963,955, filed on Apr. 26, 2018.
(60) Provisional application No. 62/591,548, filed on Nov. 28, 2017.
(60) Provisional application No. 62/509,015, filed on May 19, 2017.

(51) Int. Cl. A61K 38/13; A61P 27/02; A61K 9/107; A61K 31/573; A61P 31/00; A61P 5/44; A61P 29/00

(52) U.S. Cl. CPC A61K 38/13; A61P 27/02; A61K 9/1075; A61P 29/00; A61P 31/00; A61P 5/44; A61K 31/573

(57) ABSTRACT

The present invention relates to an ophthalmic composition comprising at least two active pharmaceutical ingredients. In particular, the active pharmaceutical ingredients are selected from the group consisting of: an alpha 2 adrenergic receptor agonist; a beta-adrenergic receptor agonist; an immunosuppressant; a lymphocyte associated antigen antagonist; an anti-inflammatory; a beta-blocker; a prostaglandin analog; a histamine receptor antagonist; a carbonic anhydrase inhibitor; and an antibiotic. In some embodiments, the composition of the invention is a nanoemulsion formulation. In one particular embodiment, the first active pharmaceutical ingredient is an alpha 2 adrenergic receptor agonist. The present invention also provides a method for treating various clinical conditions associated with an eye disorder or eye disease using the composition of the invention.

### CROSS-REFERENCE TO RELATED APPLICATIONS

This is continuation-in-part application of U.S. patent application Ser. No. 15/963,955, filed Apr. 26, 2018, which claims the priority benefit of U.S. Provisional Application Nos. 62/509,015, filed May 19, 2017, and 62/591,548, filed Nov. 28, 2017, all of which are incorporated herein by reference in their entirety.

### FIELD OF THE INVENTION

The present invention relates to an ophthalmic composition comprising at least two active pharmaceutical ingredients. In particular, the active pharmaceutical ingredients are selected from the group consisting of: an alpha 2 adrenergic receptor agonist; a beta-adrenergic receptor agonist; an immunosuppressant; a lymphocyte associated antigen antagonist; an anti-inflammatory; a beta-blocker; a prostaglandin analog; a histamine receptor antagonist; a carbonic anhydrase inhibitor; and an antibiotic. In some embodiments, the composition of the invention is a nanoemulsion formulation. The present invention also provides a method for treating various clinical conditions associated with an eye disorder or eye disease using the composition of the invention.

### BACKGROUND OF THE INVENTION

Dry eye syndrome is a multifactorial disease. Dry eye syndromes engender inflammation and ocular surface irritation. Thus, the goals for the treatment of dry eye syndrome are to improve the patient's ocular comfort and to return the ocular surface and tear composition to their basal and healthy states.

Conventional treatments for dry eye syndrome include (i) instillation of artificial tears for tear supplementation and stimulation and (ii) the use of anti-inflammatory drugs to reduce ocular surface inflammation. Generally, current dry eye treatment involves topical application of artificial tear products/lubricants, tear retention management, stimulation of tear secretion, topical application of antibiotics (e.g., erythromycin or bacitracin ointments), oral administration of tetracyclines (e.g., tetracycline, doxycycline, or minocycline), application of a calcineurin inhibitor immunosuppressant, such as cyclosporine, and corticosteroids. These treatments are often time consuming, frustrating, and frequently ineffective or variably effective.

One currently available formulation for treating dry eye syndrome is cyclosporine (commercially available as Restasis® (Allergan, Irvine, Calif.)). While cyclosporine reduces symptoms of dry eye syndrome to some extent, it has many undesired side effects, such as burning and stinging sensations. To decrease local side effects and to enhance the patient's comfort is one of the objectives of the present invention. Another product for treating dry eye syndrome is lifitegrast (commercially available as Xiidra® ophthalmic solution 5%, Shire US Inc., Lexington, Mass.). Lifitegrast (chemical name: N-{[2-(1-Benzofuran-6-ylcarbonyl)-5,7-dichloro-1,2,3,4-tetrahydro-6-isoquinolinyl]carbonyl}-3-(methyl sulfonyl)-L-phenylalanine) ophthalmic solution 5.0% has been reported to improve symptoms of ocular discomfort and eye dryness compared with placebo when administered twice daily (Sheppard et al., Ophthalmology, 2014, 121(2), pp. 475-483).

Dry eye disease (“DED”) is a multifactorial disease of ocular surface leading to discomfort, visual disturbance, and tear film instability with damage to the ocular surface. DED is typically categorized into two groups: 1) aqueous tear deficient DED and 2) evaporative DED. DED is often a result of changes to the lacrimal functional unit, or LFU. The LFU is composed of the lacrimal glands, cornea, eyelids, meibomian glands, conjunctiva, goblet cells and ocular nerves. The LFU is responsible for the sustained production of adequate tear film to consistently lubricate the ocular surface. Structural changes to the LFU can induce tear film instability and insufficiency, which in turn can lead to tear hyperosmolarity. Various stresses to the ocular surface such as environmental factors, infection, endogenous stress, antigens, genetic factors are identified as primary pathogenic triggering cause. Chronic osmotic stress from tear film can activate stress-associated pathways in ocular surface epithelial cells, thereby triggering a pro-inflammatory response that involves a mix of chemokines, cytokines, and matrix metalloproteinases. The subsequent maturation of antigen-presenting cells on the ocular surface leads to the migration, activation and expansion of autoreactive T cell lymphocytes as well as other leukocytic classes in the LFU. The constant recruitment of pro-inflammatory leukocytes onto the ocular surface may inflict epithelium damage in the form of small abrasions and epithelium barrier defects. These abrasions can eventually progress to superficial punctuate keratitis, squamous metaplasia, extracellular matrix (“ECM”) deposits, decreased goblet cell differentiation, increased epithelial cell turnover (epitheliopathy) and significant ocular surface nerve damage and neuropathy. Thus, the involvement of various cellular and physiological processes leading to inflammation, pain, tissue damage, hyperactive immune responses are associated with pathophysiology of DED and suggests the role of different molecular pathways.

Conventional therapies for treating DED are focused on using a single active pharmaceutical ingredient. This significantly limits modes of action and provides suboptimal benefit to the patients. Therefore, there is an unmet need for the treatment of DED to address multiple pathophysiological processes involving different modes of action.

Another significant deficiency in current treatment of DED is that there are no conventional ophthalmic formulations that allow the preferential distribution of active pharmaceutical ingredients into target lacrimal gland tissues. This inability to target lacrimal gland tissues significantly reduces the efficacy of conventional ophthalmic formulations.

Accordingly, there is an ongoing need for compositions and methods for effective treatment of dry eye syndrome as well as other eye disorders and/or diseases.

### SUMMARY OF THE INVENTION

The current invention in directed to a combination formulation consisting two or more APIs with different molecular targets for the pathogenesis of DED. Combination of two or more APIs with different mechanism of action in single formulation provides regulations of more than one molecular pathway and provides a significant benefit to DED patients. In particular, some embodiments of the present invention is directed to a selection of APIs in combination formulations targeting multiple molecular pathways involved in pathophysiology ocular surface diseases, anterior segment of eye diseases as well as pain and inflammation associated with ocular surgery.

One particular aspect of the present invention provides an ophthalmic composition comprising at least two active pharmaceutical ingredients. In one embodiment, the at least two active pharmaceutical ingredients are selected from a different classification. In one particular embodiment, the active pharmaceutical ingredients are selected from the following classification of active pharmaceutical ingredients: an alpha 2 adrenergic receptor agonist; a beta-adrenergic receptor agonist; an immunosuppressant; a lymphocyte associated antigen antagonist; an anti-inflammatory; a beta-blocker; a prostaglandin analog; a histamine receptor antagonist; a carbonic anhydrase inhibitor; and an antibiotic.

In some embodiments, the composition of the invention is a nanoemulsion formulation. Compared to non-nanoemulsion formulations, a nanoemulsion formulation of the invention provides a wide variety of surprising an unexpected benefits including, but not limited to, extended release of active pharmaceutical ingredients, better penetration profile of the active pharmaceutical ingredient(s) to the desired cells, increased resident time in the cornea, etc.

In one particular embodiment, the composition of the invention is an ophthalmic nanoemulsion formulation. In some embodiments, a first active pharmaceutical ingredient is said alpha 2 adrenergic agonist, and a second active pharmaceutical ingredient is selected from the group consisting of: said immunosuppressant, said lymphocyte associated antigen agonist, said corticosteroid, said beta-blocker, said prostaglandins analog, said carbonic anhydrase inhibitor, and a combination thereof. In one particular embodiment, the alpha 2 adrenergic agonist comprises brimonidine, a pharmaceutical salt thereof, or a combination thereof.

In some embodiments, the immunosuppressant is selected from the group consisting of cyclosporine, tacrolimus, and a combination thereof. Yet in other embodiments, the lymphocyte associated antigen agonist comprises Lifitegrast. Still in other embodiments, the corticosteroid is selected from the group consisting of prednisolone, methylprednisolone, difluprednate, prednisone acetate, prednisolone sodium phosphate, triamcinolone, fluocinolone; fluorometholone, betamethasone, medrysone, and a combination thereof. In other embodiments, the anti-inflammatory is selected from a group consisting of a corticosteroid, a non-steroidal anti-inflammatory drug (“NSAID”), thymosin beta 4, and a combination thereof. In one particular instances, the NSAID is selected from the group consisting of diclofenac, flubiprofen, ketorolac, ketorolac thromethamine, bromfenac, nepafenac, flurbiprofen, and a combination thereof. In yet another embodiment, the beta-adrenergic receptor agonist is selected from the group consisting of Dopexamine, Epinephrine, Isoprenaline, isoproterenol, levalbuterol, Salbutamol, albuterol, and a combination thereof. Still in other embodiments, the beta-blocker is selected from the group consisting of Timolol, Propranolo, Sotalol, nadolol, and a combination thereof. Yet still in other embodiments, the prostaglandins analog is selected from the group consisting of latanoprost, bimatoprost, travoprost, tafluprost, and a combination thereof. Yet in other embodiments, the carbonic anhydrase inhibitor is selected from the group consisting of dorzolamide, methazolamide, brinzolamide, dichlorphenamide, and a combination thereof.

In other embodiments, the active pharmaceutical ingredients consist of (i) brimonidine, a pharmaceutically acceptable salt thereof, or a combination thereof; (ii) cyclosporine; and (iii) Lifitegrast or Loteprednol.

One particular aspect of the invention provides an aqueous ophthalmic solution comprising (i) brimonidine, a pharmaceutically acceptable salt thereof, or a combination thereof and (ii) cyclosporine. In some embodiments, the aqueous ophthalmic solution is a nanoemulsion solution.

Compositions of the invention can also include a pharmaceutically acceptable excipient. In one particular embodiment, the pharmaceutically acceptable excipient comprises: an emulsion stabilizing polymer, a water-soluble polymer, a surfactant, a tonicity modifier or a stabilizer, a viscosity modifier, or a combination thereof. In one particular embodiment, the pharmaceutically acceptable excipient comprises (i) an emulsion stabilizing polymer; (ii) a surfactant; (iii) a tonicity modifier or a stabilizer selected from the group consisting of a polyol, a non-reducing disaccharide and a combination thereof or (iv) a combination thereof. In other embodiments, the pharmaceutically acceptable excipient comprises polysorbate 80, Pemulen®, carbomer copolymer type A, a polyol or a combination thereof. Yet in other embodiments, the pharmaceutically acceptable excipient comprises polysorbate 80, Pemulen®, carbomer copolymer type B, a polyol or a combination thereof. Still in other embodiments, the tonicity modifier or a stabilizer is selected from the group consisting of a polyol, a non-reducing disaccharide, and a combination thereof. Yet still in other embodiments, the viscosity modifier is selected from the group consisting of carbomer homopolymer type A, carbomer homopolymer type B, carbomer homopolymer type C, and a combination thereof. Still yet in other embodiments, the surfactant is selected from the group consisting of: (i) a nonionic surfactant, such as glyceryl laurate, polysorbate, spans, poloxamers, Nonoxynol-9; (ii) a cationic surfactant such as benzalkonium chloride, benzethonium chloride, benzododecinium bromide, cetrimonium bromide, cetrimonium chloride, tetramethylammonium hydroxide, lauralkonium chloride; (iii) a zwitterionic surfactant such as lecithin; and (iv) a combination thereof. Still in other embodiments, the pharmaceutically acceptable excipient comprises (i) an emulsion stabilizing polymer, (ii) a surfactant, (iii) a tonicity modifier or a stabilizer selected from the group consisting of a polyol, a non-reducing disaccharide, and a combination thereof, or (iv) a combination thereof.

Still another aspect of the invention provides a method for treating a clinical condition associated with eye, such as an eye disorder or an eye disease. The method includes administering to a subject in need of such a treatment a therapeutically effective amount of a composition of the invention. In some embodiments, the clinical condition associated with eye is selected from the group consisting of dry eye syndrome (e.g., keratoconjunctivitis sicca), Sjogren's syndrome, congenital alacrima, xerophthalmia (dry eye from vitamin A deficiency), keratomalacia, thyroid eye disease, ocular rosacea, eyelid disorders, meibomian gland disease, meibomian gland dysfunction, ectropion, blepharitis, blepharochalasis, sarcoidosis, stye, hordeolum, chalazion, ptosis, pterygium, eyelid edema, eyelid dermatitis, trichiasis, madarosis, dacryoadenitis, stevens-johnson syndrome, ocular graft versus host disease, dacryocystitis, conjunctivitis, keratoconjunctivitis, blepharoconjunctivitis, blepharokeratoconjunctivitis, allergic conjunctivitis, vernal conjunctivitis, conjunctival suffusion, conjunctivochalasis, subconjunctival hemorrhage, pterygium, pinguecula, chemosis, iritis, iridocyclitis, anterior uveitis, glaucoma, ocular hypertension, red eye, keratitis, scleritis, episcleritis, peripheral ulcerative keratitis, neurotrophic keratitis, neurotrophic eye disease, corneal ulcer, ulcerative keratitis, corneal abrasion, photokeratitis, ultraviolet keratitis, exposure keratitis, superficial punctuate keratitis, thygeson's superficial punctuate keratopathy, herpes zoster keratitis, acne rosacea, corneal neovascularization, corneal dystrophy, epithelial basement membrane dystrophy, fuch's dystrophy, posterior polymorphous corneal dystrophy, macular corneal dystrophy, cyclitis, uveitis, iritis, post-operative inflammation following ocular surgery (i.e. eyelid surgery, cataract surgery, corneal surgery, refractive surgery including photorefractive keratectomy, glaucoma surgery, lacrimal gland surgery, conjunctival surgery, eye muscle surgery), ocular surface conditions caused by chemical burns, thermal burns or physical trauma, ocular conditions caused by the following autoimmune or vascular disorders: rheumatoid arthritis, juvenile rheumatoid arthritis, ankulosing spondylitis, reiter's syndrome, enteropathic arthritis, psoriatic arthritis, discoid and systemic lupus erythematosus, multiple sclerosis, graves' disease, antiphospholipid syndrome, sarcoidosis, wegner's granulomatosis, behcet's syndrome, polyarteritis nodosa, takayasu's arteritis, dermatomyositis, psoriasis, relapsing polychondritis, vasculitis, sickle cell-anemia, type II diabetes, diabetic retinopathy, and a combination thereof.

In some embodiments, the eye disorder is selected from the group consisting of: (i) a dry eye syndrome; (ii) ocular graft-versus-host-disease; (iii) ocular rosacea; (iv) allergic conjunctivitis; (v) autoimmune ocular surface disease; (vi) thygeson's superficial punctuate keratopathy; (vii) herpes zoster keratitis; (viii) Stevens-Johnson syndrome; (ix) keratitis; (x) conjunctivitis; (xi) blepharitis; (xii) blepharochalasis; (xiii) conjunctivochalasis; (xiv) blepharoconjunctivitis; (xv) blepharokeratoconjunctivitis; (xvi) post-operative inflammation or pain from ocular surgery; (xvii) scleritis; (xviii) episcleritis; (xix) anterior uveitis; (xx) iritis; (xxi) cyclitis; (xxii) ocular surface vascular disorder; (xxiii) ulcerative keratitis; (xxiv) photokeratitis; (xxv) dacryocystitis; (xxvi) eyelid disorder; (xxvii) congenital alacrima; (xxviii) xerophthalmia; (xxix) dacryoadenitis; and (xxx) ocular surface disorder induced by chemical burns, thermal burns, use of contact lenses, or physical insult to the ocular surface.

Yet in some embodiments, the dry eye syndrome is selected from the group consisting of sjogren's syndrome, meibomian gland dysfunction and keratoconjunctivitis. Still in other embodiments, the eyelid disorder comprises eyelid inflammation, pain and/or edema.

In some embodiments, the composition is administered topically to an eye of said subject. In one embodiment, the composition is formulated as a homogeneous ophthalmic aqueous formulation. Yet in another embodiment, the composition is formulated as a heterogeneous ophthalmic aqueous solution. In some instances, the heterogeneous ophthalmic aqueous solution comprises emulsion, suspension or a combination thereof.

### BRIEF DESCRIPTION OF THE DRAWINGS

FIG. 1 is a table showing measured median diameter of particle size distribution [Dx(50)] of some of the ophthalmic formulations of the invention.

FIG. 2 is a table showing median diameter of particle size distribution [Dx(50)] of an ophthalmic nanoemulsion formulation of brimonidine tartrate and loteprednol etabonate at different temperatures (RT is room temperature) at days 0, 36 and 66.

FIG. 3 is a table shows measured median diameter of particle size distribution [Dx(50)] of (i) brimonidine tartrate (0.2% w/w) and cyclosporine (0.05% w/w) combination of ophthalmic nanoemulsion formulation and (ii) brimonidine tartrate (0.2% w/w) and cyclosporine (0.1% w/w) ophthalmic nanoemulsion formulation at different temperatures at days 0 and 14.

FIG. 4 is a table showing partitioning of brimonidine tartrate and cyclosporine in various phases, oil and aqueous, of (i) combination of brimonidine tartrate (0.2% w/w) and 0.05% w/w cyclosporine ophthalmic nanoemulsion formulation and (ii) combination of brimonidine tartrate (0.2% w/w) and 0.1% w/w cyclosporine ophthalmic nanoemulsion formulation.

FIG. 5 shows stability data at different temperatures of an ophthalmic nanoemulsion formulation of the present invention consisting of active pharmaceutical ingredients brimonidine tartrate and cyclosporine.

FIG. 6A is a graph showing particle size distribution [Dx(50)] of an ophthalmic nanoemulsion formulation of the present invention having active pharmaceutical ingredients brimonidine tartrate and loteprednol etabonate.

FIG. 6B is a graph showing particle size distribution [Dx(50)] of an ophthalmic nanoemulsion formulation of the present invention having active pharmaceutical ingredients brimonidine tartrate (0.2% w/w) and 0.05% w/w cyclosporine.

FIG. 6C is a graph showing particle size distribution [Dx(50)] of an ophthalmic nanoemulsion formulation of the present invention having active pharmaceutical ingredients (B) brimonidine tartrate (0.2% w/w) and 0.1% w/w cyclosporine.

### DETAILED DESCRIPTION OF THE INVENTION

One aspect of the invention provides a nanoemulsion ophthalmic composition comprising at least two active pharmaceutical ingredients. As used herein, the term “nanoemulsion” refers to emulsion having a median emulsion droplet particle size (i.e., Dx(50)) of about 250 nm or less, typically about 220 nm or less, often about 200 nm or less, and most often about 100 nm or less.

In one particular embodiment, the active pharmaceutical ingredients are selected from the following classification of active pharmaceutical ingredients: an alpha 2 adrenergic receptor agonist; a beta-adrenergic receptor agonist; an immunosuppressant; a lymphocyte associated antigen antagonist; an anti-inflammatory; a beta-blocker; a prostaglandin analog; a histamine receptor antagonist; a carbonic anhydrase inhibitor; and an antibiotic. In one particular embodiment, the composition of the invention comprises an alpha 2 adrenergic receptor agonist in combination with one or more of a calcineurin inhibitor (e.g., cyclosporine) and a lymphocyte function-associated antigen antagonist (e.g., lifitegrast). In another embodiment, the composition of the invention comprises an alpha 2 adrenergic receptor agonist in combination with a corticosteroid. Still another aspect of the invention provides a composition comprising an alpha 2 adrenergic receptor agonist in combination with one or more of the following components: (i) a calcineurin inhibitor; (ii) a lymphocyte function-associated antigen antagonist; (iii) an anti-inflammatory (e.g., corticosteroid comprising loteprednol, thymosin beta 4, etc.); (iv) a sodium channel blocker; (v) a non-steroidal anti-inflammatory drug (i.e., NSAID); and (vi) an antihistamine.

In some embodiments, the anti-inflammatory is a corticosteroid. Still in other embodiments, the anti-inflammatory is an NSAID. Yet in other embodiments, the anti-inflammatory is thymosin beta 4.

Yet in some embodiments, compositions of the invention are heterogeneous solution formulations, containing a combination of therapeutically effective amount of active pharmaceutical components in the formulation. In other embodiments, compositions of the invention are homogeneous aqueous formulations, containing a combination of therapeutically effective amount of active pharmaceutical components in the formulation.

The compositions or formulations of the invention can contain just two active ingredients or more than two active ingredients. In some embodiments, at least one of the active ingredient is an alpha 2 adrenergic receptor agonist such as brimonidine, or a pharmaceutically acceptable salt there of or a combination thereof.

Compositions of the invention are useful for treatment of various eye disorders or eye diseases including, but not limited to, dry eye syndrome (keratoconjunctivitis sicca), Sjogren's syndrome, congenital alacrima, xerophthalmia (dry eye from vitamin A deficiency), keratomalacia, thyroid eye disease, ocular rosacea, eyelid disorders, meibomian gland disease, meibomian gland dysfunction, ectropion, blepharitis, blepharochalasis, sarcoidosis, stye, hordeolum, chalazion, ptosis, pterygium, eyelid edema, eyelid dermatitis, trichiasis, madarosis, dacryoadenitis, stevens-johnson syndrome, ocular graft versus host disease, dacryocystitis, conjunctivitis, keratoconjunctivitis, blepharoconjunctivitis, blepharokeratoconjunctivitis, allergic conjunctivitis, vernal conjunctivitis, conjunctival suffusion, conjunctivochalasis, subconjunctival hemorrhage, pterygium, pinguecula, chemosis, iritis, iridocyclitis, anterior uveitis, glaucoma, ocular hypertension, red eye, keratitis, scleritis, episcleritis, peripheral ulcerative keratitis, neurotrophic keratitis, neurotrophic eye disease, corneal ulcer, ulcerative keratitis, corneal abrasion, photokeratitis, ultraviolet keratitis, exposure keratitis, superficial punctuate keratitis, thygeson's superficial punctuate keratopathy, herpes zoster keratitis, acne rosacea, corneal neovascularization, corneal dystrophy, epithelial basement membrane dystrophy, fuch's dystrophy, posterior polymorphous corneal dystrophy, macular corneal dystrophy, cyclitis, uveitis, iritis, post-operative inflammation following ocular surgery (i.e. eyelid surgery, cataract surgery, corneal surgery, refractive surgery including photorefractive keratectomy, glaucoma surgery, lacrimal gland surgery, conjunctival surgery, eye muscle surgery), ocular surface conditions caused by chemical burns, thermal burns or physical trauma, ocular conditions caused by the following autoimmune or vascular disorders: rheumatoid arthritis, juvenile rheumatoid arthritis, ankulosing spondylitis, reiter's syndrome, enteropathic arthritis, psoriatic arthritis, discoid and systemic lupus erythematosus, multiple sclerosis, graves' disease, antiphospholipid syndrome, sarcoidosis, wegner's granulomatosis, behcet's syndrome, polyarteritis nodosa, takayasu's arteritis, dermatomyositis, psoriasis, relapsing polychondritis, vasculitis, sickle cell-anemia, type II diabetes, diabetic retinopathy, and a combination thereof.

Some aspects of the invention provide a method for treating dry eye syndrome using the compositions disclosed herein. There are two major classes of dry eye syndrome: (i) aqueous tear-deficient dry eye (ADDE) and (ii) evaporative dry eye (EDE). There are also cases of mixed mechanism dry eye (i.e., both ADDE and EDE). ADDE is primarily due to failure of lacrimal tear secretion. ADDE can be further subdivided into Sjogren syndrome dry eye (where the lacrimal and salivary glands are targeted by an autoimmune process, e.g., rheumatoid arthritis) and non-Sjogren's syndrome dry eye (lacrimal dysfunction, but the systemic autoimmune features of Sjogren's syndrome are excluded, e.g., age-related dry eye). In contrast, EDE is primarily due to excessive water loss from the exposed ocular surface in the presence of normal lacrimal secretory function. Its causes can be extrinsic (e.g., ocular surface disorder due to some extrinsic exposure, contact lens wear or vitamin A deficiency) or intrinsic (e.g., Meibomian gland dysfunction and disorders of eyelid aperture). Meibomian glands secrete a mixture of lipids and other components that form the outer layer of the preocular tear film. This lipid layer functions to decrease tear film evaporation. Meibomian gland dysfunction (MGD) leads to evaporative dry eye disease. One of the most well recognized clinic finding in MGD is the presence of numerous telangiectatic blood vessels coursing across the eyelid margin. MGD can also accompany tear deficient dry eye disease, as seen in ocular graft-versus-host-disease (oGVHD). Other specific dry eye syndromes that can be treated using compositions of the invention include keratoconjunctivitis, dry eye caused by conjunctivitis, dry eye caused by allergic conjunctivitis, dry eye caused by blepharitis, dry eye caused by keratitis, dry eye caused by dacryoadenitis, dry eye caused by ocular rosacea, dry eye caused by boehm syndrome, dry eye caused by conjunctivochalasis, dry eye caused by blepharoconjunctivitis, dry eye caused by blepharokeratoconjunctivitis, dry eye caused by superficial punctuate keratitis, dry eye caused by thygeson's superficial punctuate keratopathy, dry eye caused by oGVHD, Sjogren's dry eye syndrome, dry eye caused by Stevens-Johnson syndrome, MGD, dry eye caused by meibomian gland disease, vitamin A deficiency induced dry eye, pharmacological induced dry eye (i.e. hormone replacement therapy, blood pressure medication, antihistamine, antidepressants, anticholinergic medications, glaucoma medication, antihypertensives, diuretics, sedatives, isotretinoin, nasal decongestants, oral contraceptives, beta-blockers, phenothiazines, atropine, pain relieving opiates), pregnancy induced dry eye, LASIK surgery or refractive surgery induced dry eye, dry eye induced by collagen vascular diseases (i.e. systemic lupus erythematosus, Wegener's granulomatosis, rheumatoid arthritis, relapsing polychondritis), dry eye caused by the infiltration of the lacrimal glands by tumors or sarcoidosis, dry eye caused by postradiation fibrosis of tear producing glands, dry eye caused by lacrimal gland, meibomian gland, or goblet cell ablation, dry eye caused by sensory denervation, dry eye caused by thermal or chemical burns, dry eye caused by underlying diabetic conditions, dry eye caused by viral, fungal, or bacterial infection, dry eye caused by prolonged contact lens use, dry eye caused by eyelid disorders or injury to the eyelid (i.e. bulging eyes, drooping eyelid), dry eye caused by corneal dystrophy, dry eye caused by autoimmune disorders, age-induced dry eye, and a combination thereof.

In some embodiments, methods for treating dry eye syndrome comprise treating a patient in need of a treatment for Meibomian gland dysfunction (MGD). In other embodiments, methods for treating dry eye syndrome comprise treating a patient in need of a treatment for aqueous tear-deficient dry eye (ADDE). In some instances, methods for treating ADDE comprise treating a patient in need of a treatment for Sjogren dry eye syndrome, ocular Graft-Versus-Host-Disease (oGVHD) or non-Sjogren dry eye syndrome. Yet in other embodiments, methods for treating dry eye syndrome comprise treating a patient in need of a treatment of evaporative dry eye (EDE). Still in other embodiments, methods of the invention include treating a patient in need of a treatment for mixed mechanism dry eye consisting of ADDE and EDE. Yet still in other embodiments, methods of the invention include treating a patient suffering from dry eye syndrome due to a complication of refractive eye surgery, or is attributable to one or more of the following causes: vitamin A deficiency, ocular surface disorders, allergy, aging, contact lens usage, medication usage or eyelid disorders.

In some embodiments, compositions of the invention include an alpha 2 (α2) adrenergic receptor agonists. Exemplary alpha 2 adrenergic receptor agonists include, but are not limited to, brimonidine, 4-NEMD, 7-Me-marsanidine, agmatine, apraclonidine, cannabigerol, clonidine, detomidine, dexmedetomidine, fadolmidine, guanabenz, guanfacine, lofexidine, marsanidine, medetomidine, methamphetamine, mivazerol, rilmenidine, romifidine, talipexole, tizanidine, tolonidine, xylazine, xylometazoline, and the like including pharmaceutically acceptable salts thereof. In one particular embodiment, the alpha 2 adrenergic receptor agonist is brimonidine (5-Bromo-N-(4,5-dihydro-1H-imidazol-2-yl) quinoxalin-6-amine), a pharmaceutically acceptable salt thereof or a combination thereof.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, trifluoroacetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. Particular examples of pharmaceutically acceptable salts of brimonidine include, but not limited to, tartrate salt, trifluoroacetate salt, hydrochloric salt, acetate salt, oxalic acid salt, as well as others disclosed herein and/or known to one skilled in the art.

Alpha-2 adrenergic receptor agonists are those compounds that activate alpha-2 adrenergic receptors. There are three subtypes of this receptor, designated A, B, and C. An alpha-2 adrenergic receptor agonist that can activate any or all of these receptor subtypes can be used in the present invention. However, in some embodiments of the invention, an alpha 2 adrenergic receptor agonist has a higher activity or efficacy at the alpha-2A adrenergic receptor subtype compared to its activity at the alpha-2B receptor subtype (e.g., brimonidine and its salts). In some embodiments, the alpha 2 adrenergic agonist in compositions of the invention has a higher alpha 2A agonist activity compared to alpha 2B agonist activity. In some instances, the alpha 2A agonist activity of the alpha 2 adrenergic agonist is at least about 10% greater, typically at least about 20% greater and often at least about 30% greater than its alpha 2B agonist activity. As used herein, the term “about” when referring to a numeric value means±20%, typically ±10%, often ±5% and most often ±2% of the numeric value.

In one embodiment, the second pharmaceutically active compound comprises a calcineurin inhibitor, a lymphocyte function-associated antigen antagonist, or a combination thereof. Calcineurin (CaN) is a calmodulin and calcium dependent serine/threonine protein phosphatase (also known as protein phosphatase 3, and calcium-dependent serine-threonine phosphatase). It activates the T cells of the immune system and can be blocked by a class of drugs called calcineurin inhibitors, which includes cyclosporine, tacrolimus, pimecrolimus, voclosporin, as well as others known to one skilled in the art. Any known calcineurin inhibitors (e.g., cyclosporine) or those developed by one skilled in the art can be used in compositions of the invention. Lymphocyte function-associated antigen (LFA)-1/intercellular adhesion molecule (ICAM)-1 interactions mediate several important steps in the evolution of an immune response. Exemplary lymphocyte function-associated antigen antagonists include, but are not limited to, lifitegrast (i.e., (S)-2-(2-(benzofuran-6-carbonyl)-5,7-dichloro-1,2,3,4-tetrahydroisoquinoline-6-carboxamido)-3-(3-(methylsulfonyl)phenyl)propanoic acid), which is a water-soluble drug that blocks LFA-1 from binding to ICAM-1, and other lymphocyte function-associated antigen antagonists that are known to one skilled in the art. Any lymphocyte function-associated antigen can be used in compositions of the invention.

Yet in another embodiment, the composition of the invention is formulated as an aqueous solution, which can be a homogeneous or heterogeneous solution. In such embodiments, compositions of the invention include at least two active ingredients. In one particular embodiment, the formulation contains one active ingredient that is water soluble and the another active ingredient that is a lipophilic. In another particular embodiment, the formulation contains two or more active ingredients that are water soluble. In another particular embodiment, the formulation contains two or more active ingredients that are lipophilic. Still in other embodiments, compositions of the invention are formulated as an aqueous ophthalmic solution. As stated above, the aqueous ophthalmic solution can be homogenous or heterogeneous and can include aqueous suspension or dispersion, where at least some of the active ingredients are present as suspension or dispersion in aqueous solution. The aqueous ophthalmic solution can be a substantially homogeneous aqueous solution, where substantially all (i.e., ≥85%, typically ≥90%, often ≥95%, and most often ≥97%) of the active ingredients are dissolved in the aqueous solution.

In one particular embodiment, one of the active ingredients is brimonidine, its pharmaceutically acceptable salt thereof or a combination thereof. The composition includes a second active ingredient that can be cyclosporine, lifitegrast or a combination thereof.

As used herein, the terms “active ingredient” and “active pharmaceutical ingredient” are used interchangeably herein and refer to a compound that is used or known to one skilled in the art in treating an eye disorder, such as dry eye syndrome. Thus, while water and oil can be present in some formulations, they are not used primarily for the purpose of treating eye disorder but are used as a vehicle to carry active ingredients. Generally, an active ingredient works on a particular receptor or cells or has been approved for treating an eye disorder by the U.S. Food and Drug Administration (“FDA”).

In one particular embodiment, the term “active ingredient” refers to an alpha 2 adrenergic receptor agonist; a beta-adrenergic receptor agonist; an immunosuppressant; a lymphocyte associated antigen antagonist; an anti-inflammatory (e.g., corticosteroid, NSAID, thymosin beta 4, etc.); a beta-blocker; a prostaglandin analog; a histamine receptor antagonist; a carbonic anhydrase inhibitor; and an antibiotic. Yet in another embodiment, the term “active ingredient” refers to: an alpha 2 adrenergic agonist; a calcineurin inhibitor; a lymphocyte function-associated antigen antagonist; a corticosteroid; CRGP receptor antagonists; anti-CGRP receptor monoclonal antibodies; an inhibitor of adrenomedullin, serotonin, cathelicidin or neuropeptides; a sodium channel blocker; an antihistamine; and/or a non-steroidal anti-inflammatory drug. Other ingredients that may be present in formulations/compositions of the invention are used primarily as pharmaceutically acceptable excipients or vehicles, such as a pH adjusting agent, a tonicity modifier or a stabilizer, a surfactant, an emulsion stabilizer, etc.

A therapeutically effective amount of an active ingredient in the composition of the invention can be readily determined by one skilled in the art. In some embodiments, the composition of the invention is formulated as a heterogeneous aqueous solution. In one particular embodiment, the composition of the invention include from about 0.01 to about 5 mg/mL (about 0.001% to about 0.5% w/v) typically about 0.2% w/v or less (e.g., 0.05-0.2% often 0.07-0.15%) of brimonidine or a salt thereof (e.g., brimonidine tartrate and hydroxy brimonidine trifluoroacetate). The ingredient amounts are presented in units of either % weight/volume (% w/v) or weight/weight (% w/w). In one specific embodiment, brimonidine tartrate is used as an alpha 2 adrenergic agonist. In one embodiment, the amount of brimonidine tartrate present in the composition is from about 0.01% w/w to about 1% w/w, typically from 0.01% w/w to about 0.7% w/w, and often from about 0.02% to about 0.5% w/w.

In some embodiments, the second therapeutically active compound comprises cyclosporine. In one particular embodiment, the second therapeutically active compound comprises cyclosporine A. A typical amount of cyclosporine A present in the composition of the invention is from about 0.005% w/w to about 0.5% w/w, often from about 0.01% w/w to about 0.3% w/w.

Still in another embodiment, the second therapeutically active compound comprises Lifitegrast®. In one particular embodiment, the amount of lifitegrast present in compositions of the invention is from about 0.1% w/w to about 20% w/w, typically from about 0.2% w/w to about 15% w/w, and often from about 0.3% w/w to about 10% w/w.

Yet in some embodiments, the second therapeutically active compound comprises a corticosteroid. Exemplary corticosteroids include, but are not limited to, methylprednisolone, hydrocortisone, betamethasone, dexamethasone and loteprednol etabonate. In one particular embodiment, the corticosteroid used in the composition of the invention is loteprednol etabonate. In some embodiments, the amount of loteprednol etabonate present in the compositions of the invention is from about 0.01% w/w to 2% w/w; typically from about 0.05% w/w to 1%, and often from about 0.1% to about 0.3%.

Still in another embodiment, the second therapeutically active compound comprises a sodium channel blocker and/or mucolytic agents. Suitable sodium channel blockers and/or mucolytic agents for treatment of eye disorder are known to one skilled in the art and include those disclosed, for example, in U.S. Pat. Nos. 9,586,911, 9,346,753, 8,980,898, 8,673,340, 8,058,278, 7,875,619, 7,868,010, 7,842,697, 7,820,678, 7,410,968, 7,399,766, 7,388,013, 7,375,107, 7,368,451, 7,368,450, 7,368,447, 7,375,107, 7,368,451, 7,368,447, 7,345,044, 7,332,496, 7,317,013, 7,247,637, 7,247,636, 7,241,766, 7,192,959, 7,192,958, 7,189,719, 7,186,833, 7,064,129, 7,030,177, 7,026,325, 6,995,160, 6,903,105, 6,858,615, and 6,858,614 which are incorporated herein by reference in their entirety. Specific examples of suitable sodium channel blockers for the present invention include, but are not limited to amiloride, benzamil, phenamil, amiloride analogues, as well as those disclosed in U.S. Pat. Nos. 9,586,911, 9,346,753, 8,980,898, 8,673,340, 8,058,278, 7,875,619, 7,868,010, 7,842,697, 7,820,678, 7,410,968, 7,399,766, 7,388,013, 7,375,107, 7,368,451, 7,368,450, 7,368,447, 7,375,107, 7,368,451, 7,368,447, 7,345,044, 7,332,496, 7,317,013, 7,247,637, 7,247,636, 7,241,766, 7,192,959, 7,192,958, 7,189,719, 7,186,833, 7,064,129, 7,030,177, 7,026,325, 6,995,160, 6,903,105, 6,858,615, and 6,858,614.

Yet still in another embodiment, the second therapeutically active compound comprises a non-steroidal anti-inflammatory drug (i.e., NSAID). Suitable NSAIDs that are useful in treating eye disorder include ketorolac (0.05 to 0.3%), diclofenac (0.01 to 1%), flurbiprofen (0.01 to 1%), bromfenac (0.01 to 0.5%), nepafenac (0.05 to 0.5%), etc. Some of these are commercially available as Acular, Acular PF, and Acular LS (ketorolac tromethamine, Allergan), Ocufen (flurbiprofen sodium, Allergan), Voltaren (diclofenac sodium, Novartis), Xibrom (bromfenac ophthalmic solution, Ista Pharmaceuticals), Prolensa (bromfenac ophthalmic solution, Bausch & Lomb) and Nevanac (nepafenac, Alcon).

In still further embodiments, the second therapeutically active compound comprises an antihistamine. Suitable antihistamines that are useful in treating eye disorder include alcaftadine (0.01 to 0.5%), azelastine (0.001% to 0.2%), bepotastine (0.1% to 3%), emedastine (0.001% to 0.2%), epinastine (0.001% to 0.2%), ketotifen (0.001% to 0.2%), and olopatadine (0.01% to 1.5%). Some of these are commercially available as Lastacaft (alcaftadine, Allergan), Optivar (zelastine hydrochloride, Meda Pharmaceuticals), Bepreve (bepotastine besilate, Bausch & Lomb), Emadine (emedastine difumarate, Alcon), Elestat (epinastine hydrochloride, Allergan), Alaway (ketotifen fumarate, Baush & Lomb), Zaditor (ketotifen fumarate, Alcon), Pazeo (olopatadine hydrochloride, Alcon) Pataday (olopatadine hydrochloride, Alcon), and Patanol (olopatadine hydrochloride, Alcon).

Still in other embodiments, the second therapeutically active compound comprises thymosin beta 4. In some embodiments, the amount of thymosin beta 4 present in the compositions of the invention is from about 0.01% w/w to 2% w/w; typically from about 0.05% w/w to 1%, and often from about 0.0.05% to about 0.3%.

In other embodiments, the second therapeutically active compound comprises a prostaglandin analog. Exemplary prostaglandin analogs that are useful in compositions of the invention include, but are not limited to, latanoprost, bimatoprost, travoprost, and tafluprost. When present, the amount of prostaglandin analog in the compositions of the invention is from about 0.001% w/w to 1% w/w; typically from about 0.005% w/w to 0.5%, and often from about 0.005% to about 0.1%.

In some embodiments, compositions of the invention are used as an ophthalmic formulation. Such ophthalmic formulations can be homogeneous or heterogeneous formulations. In such embodiments, the formulated composition contains an oil or a fatty acid ester. A fatty acid ester has the meaning commonly understood in the art, being an ester formed between an alcohol and a fatty acid. Exemplary fatty acid esters that are useful in formulations of the invention include, but are not limited to, triglyceride esters commonly known as vegetable oils, mono and diglyceride esters of fatty acids, fatty acid methyl esters, as well as other fatty acid esters that are known to one skilled in the art. It should be appreciated the fatty acid ester can be a mixture of several chemical compounds or an essentially pure compound. Typically, the fatty acid ester is a vegetable oil. Particular examples of vegetable oils that can be used include, but are not limited to, castor oil, sesame oil, soybean oil, cottonseed oil, olive oil, peanut oil, safflower oil, sunflower oil, palm oil, palm kernel oil, canola oil, and Miglyol Oil® In one particular embodiment, the fatty acid ester is castor oil.

Various vehicles can be used in the ophthalmic formulations of the present invention. These vehicles include, but are not limited to, purified water (water), polyvinyl alcohol, povidone, hydroxypropyl methyl cellulose, poloxamers, carboxymethyl cellulose, hydroxyethyl cellulose, cyclodextrin and a mixture of two or more thereof. The vehicle is used in the formulation in amounts as needed to provide the concentration of the active compound(s) disclosed herein. In one particular embodiment, the vehicle comprises water.

In some embodiments of this invention, an emulsion stabilizing polymer is used. While not intending to limit the scope of the invention, emulsion stabilizing polymers generally contain hydrophilic groups such as cellulose, sugars, ethylene oxide, hydroxide, carboxylic acids or other polyelectrolytes. Without being bound by any theory, it is believed that these polymers help to stabilize emulsions by increasing the viscosity of the formulation as well as by reducing the interfacial tension. Some examples of emulsion stabilizing polymers useful in this invention include, but are not limited to, carbomers, Pemulen®, sodium carboxymethylcellulose, hydroxypropylmethylcellulose, povidone, polyvinyl alcohol, polyethylene glycol and a mixture of two or more thereof.

In one particular embodiment, Pemulen (B.F. Goodrich, Cleveland, Ohio) is used as the polymeric based stabilizer. Pemulen® are Acrylates/C₁₀₋₃₀ Alkyl Acrylate Cross-Polymers.

In another embodiment of this invention, the formulation comprises a surfactant. Without being bound by any theory, a surfactant is used to help facilitate the formation of the emulsion and improve its stability. Any type of surfactant can be used including, anionic, cationic, amphoteric, zwitterionic, nonionic, as well as a mixture of two or more thereof. In one particular embodiment, the formulation of the invention comprises a nonionic surfactant. Exemplary nonionic surfactants include, but are not limited to, polysorbates, poloxamers, alcohol ethoxylates, ethylene glycol-propylene glycol block copolymers, fatty acid amides, alkylphenol ethoxylates, phospholipids, and two or mixture thereof. In one particular embodiment, the surfactant is Polysorbate 80 (ICI Americas, Inc., Wilmington, Del.).

Various buffers and means for adjusting pH can be used so long as the resulting preparation is ophthalmically acceptable. Accordingly, useful buffers include, but are not limited to, acetate buffers, citrate buffers, phosphate buffers and borate buffers. In one particular embodiment, a buffering agent is used to maintain the pH in the therapeutically useful range of pH 4-10, typically about pH 5-8, often a pH range of 6.5-8.0, more often a pH range of 7.0-8.0, and most often a pH range of 7.2-7.6. It should be appreciated, however, that the scope of the invention is not limited to these particular pH ranges. In general, any pH range that provides suitable penetration of the active ingredient(s) through the eye can be used. Typically, a buffering agent known to those skilled in the art is used including, but not limited to, acetate, borate, tris, carbonate, citrate, histidine, succinate, and phosphate. In one particular embodiment, the buffering agent comprises boric acid. In another embodiment, the buffering agent comprises sodium citrate.

To provide the ophthalmic formulations with a pH substantially corresponding to the pH of the fluids of the eye or at an acceptable physiological pH, as described above, the pH of the ophthalmic formulation can be adjusted by addition of an acid or a base in quantity sufficient to achieve the desired pH. The pH adjustment can be achieved through use of various chemicals such as hydrochloric acid, sodium hydroxide, citric acid, sodium citrate, acetic acid, sodium acetate, ammonium acetate, succinic acid, lactic acid, calcium lactate, sodium lactate, sodium fumarate, sodium propionate, boric acid, tris base, ammonium borate, maleic acid, phosphoric acid, sulfuric acid and aluminum potassium sulfate and the like. A specific example of an acid that can be used to adjust the pH of the aqueous buffered ophthalmic formulation is 1 N hydrochloric acid. A specific example of a base that can be used to adjust the pH of the aqueous buffered ophthalmic formulation is 1 N sodium hydroxide. However, it should be appreciated that the scope of the invention is not limited to this particular acid and base. In general, any pharmaceutically acceptable acids and bases can be used to adjust the pH. In one particular embodiment, the ophthalmic formulations of the present invention contain a combination of dibasic and monobasic phosphate or boric acid and sodium borate—as buffering agents. For example, the formulations contain an amount of boric acid and sodium borate sufficient to buffer the formulation in a pH range of 6.5-8.0 or 7.5-8.0 or dibasic and monobasic phosphate sufficient to buffer the formulation in a pH range of 6.5-8.0 or 7.0-8.0 or 7.5-8.0.

In another embodiment, a tonicity agent (tonicity-adjusting agent) is used to adjust the composition of the formulation to the desired isotonic range. The tonicity-adjusting agent can be a polyol or a disaccharide including non-reducing disaccharides. Such tonicity agents are known to one skilled in the art, and include, but are not limited to, glycerin, mannitol, sorbitol, trehalose, xylitol, sodium chloride, and other electrolytes. In one particular embodiment, the tonicity agent is glycerin.

If desired, gum and/or resin can be included in the formulations of the invention, including for example, sodium polyacrylate, cellulose ether, calcium alginate, carboxyvinyl polymer, ethylene-acrylic acid copolymer, vinyl pyrrolidone polymer, vinyl alcohol-vinyl pyrrolidone copolymer, nitrogen-substituted acrylamide polymer, polyacrylamide, cationic polymer such as cationic guar gum, dimethylacrylic ammonium polymer, acrylic acid-methacrylic acid copolymer, polyoxyethylene-polypropylene copolymer, polyvinyl alcohol, pullulan, agar, gelatine, chitosan, polysaccharide from tamarindo seed, xanthan gum, carageenan, high-methoxyl pectin, low-methoxyl pectin, guar gum, acacia gum, microcrystalline cellulose, arabinogalactan, karaya gum, tragacanth gum, alginate, albumin, casein, curdlan, gellan gum, dextran, cellulose, polyethyleneimine, high polymerized polyethylene glycol, cationic silicone polymer, synthetic latex, acrylic silicone, trimethylsiloxysilicate and fluorinated silicone resin.

In some embodiments, the formulations are preservative-free. In other embodiments, a preservative is used. Preservatives are used, for example, to prevent bacterial contamination in multiple-use ophthalmic preparations. Exemplary preservatives include, but are not limited to, benzalkonium chloride, stabilized oxychloro complexes (otherwise known as Purite®), phenylmercuric acetate, chlorobutanol, benzyl alcohol, parabens, and thimerosal. In one particular embodiment, the preservative is Purite®.

Other excipient components or ingredients that can also be included in the ophthalmic formulations of the present invention are chelating agents and antibiotics. Suitable chelating agents are known in the art. Particular examples of useful chelating agents include, but are not limited to, edetate salts like edetate disodium, edetate calcium disodium, edetate sodium, edetate trisodium, and edetate dipotassium. In one particular embodiment, the chelating agent is edentate disodium. It should be appreciated that other chelating agents may also be used in place of or in conjunction with edentate disodium. Some examples of antibiotics that can be included in formulations of the invention include, but are not limited to, trimethoprim sulfate/polymyxin B sulfate, gatifloxacin, moxifloxacin hydrochloride, tobramycin, teicoplanin, vancomycin, azithromycin, clarithromycin, amoxicillin, penicillin, ampicillin, carbenicillin, ciprofloxacin, levofloxacin, amikacin, gentamicin, kanamycin, neomycin and streptomycin.

The formulations of the present invention can be packaged in various package forms known in the field of topical ophthalmics. In one embodiment, the formulation is packaged in sterile, preservative-free single-use packs or vials or containers (i.e., the unit dose vials). Each vial, for example as small as a 0.9 mL, may be made of low density polyethylene so as to contain a small quantity of the formulation, e.g., 0.4 mL for a single use. This way, where the pharmaceutical composition is sterilized and contained in disposable single-dose containers for topical use in drop form, multiple vials in the form of a set of 30 vials, 60 vials and so on can be packaged in a tray with a lid, for example, a polypropylene tray with an aluminum peelable lid. The entire contents of each tray can be dispensed intact, and one vial or pack is used each time and immediately discarded after each use. For example, plastic ampules or vials or containers can be manufactured using blow-fill-seal (BFS) technology. The BFS processes may involve plastic extrusion, molding, aseptic filling, and hermetic sealing in one sequential operation and those processes are known in the art. In another embodiment, the formulation is packaged in multi-dose vials such that the materials can be dispensed as sterile at each time using specialized container/closure maintaining the sterility integrity. In yet another embodiment, the formulation is packed in conventional vials/containers as sterile product.

In some embodiments, the dosage form of the invention is eye drops of heterogeneous aqueous solution, eye drop formulations containing two or more active ingredients in which the first active ingredient is an alpha 2 adrenergic receptor agonist and a second active ingredient is selected from the group consisting of a calcineurin inhibitor, a lymphocyte function-associated antigen antagonist, a corticosteroid, NSAID, a sodium channel blocker, an anti-histamine, and a combination of two or more thereof. For example, an eye drop formulations can contain bromonidine or brimonidine tartrate and cyclosporine, or bromonidine or brimonidine tartrate and lifitegrast or cyclosporine and lifitegrast combination or brimonidine or brimonidine tartrate, cyclosporine and lifitegrast. Eye drops typically contain, according to the invention, aqueous/oily suspensions of the active ingredients in pharmaceutically acceptable carriers and/or excipients. In some embodiments, the particle size of the active ingredient employed is about 10 μm or less, typically 5 μm or less, often 1 μm or less, more often 0.5 μm or less, still more often 0.2 μm or less and most often 0.15 μm or less

In another aspect, the invention relates to methods of treating a subject or human patient suffering from an eye disorder (e.g., dry eye syndrome) by administering to the eye of the patient an ophthalmic formulation disclosed herein. For example, in some embodiments, formulations used in treating an eye disorder comprise (i) brimonidine or a pharmaceutically acceptable salt thereof (e.g., brimonidine tartrate) and cyclosporine, (ii) brimonidine or a pharmaceutically acceptable salt thereof and lifitegrast, (iii) cyclosporine and lifitegrast combination or (iv) all three actives, i.e., brimonidine or a pharmaceutically acceptable salt thereof, cyclosporine and lifitegrast. Still other examples of compositions of the invention include brimonidine or a pharmaceutically acceptable salt thereof and loteprednol; a combination of brimonidine a pharmaceutically acceptable salt thereof and an NSAID; and a combination of brimonidine a pharmaceutically acceptable salt thereof and a sodium channel blocker and brimonidine or a pharmaceutically acceptable salt there of and an antihistamine.

The active ingredients are present in an amount effective to provide a desired therapeutic benefit to a patient suffering from an eye disorder to whom the composition is administered. The therapeutically effective amount should be sufficient to realize relief from the eye disorder after the treatment. The eye of a subject or human patient can be the entire eye structure or a tissue or gland in or around the eye such as the ocular tissue, eyelids, margin of the eyelid of the subject, ocular surface. The ophthalmological pharmaceutical formulation is topically administrable and/or is administered in, on or around the eye. The dry eye syndrome can be aqueous tear-deficient dry eye (ADDE) or evaporative dry eye (EDE) or consists of both ADDE and EDE (mixed mechanism dry eye). ADDE may be Sjogren syndrome dry eye (where the lacrimal and salivary glands are targeted by an autoimmune process, e.g., rheumatoid arthritis) and non-Sjogren's syndrome dry eye (lacrimal dysfunction, but the systemic autoimmune features of Sjogren's syndrome are excluded, e.g., age-related dry eye). The actual dose of the active compounds of the present invention depends on the specific compound, and on the condition to be treated; the selection of the appropriate dose is well within the knowledge of the skilled artisan.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

An example of a topical heterogeneous ophthalmic solution with its various components (w/w) useful for treating an eye disorder (e.g., dry eye syndrome) is as follows: brimonidine tartrate in the amount of 0.02% to 0.2% by weight, preferably about 0.075% and cyclosporine 0.01 to 0.1% by weight, surfactant such as Polysorbate 80 at about 0.02%-2% by weight or poloxamer/tyloxapol at about 0.1% and 0.25% by weight; carbomer copolymer (type A or type B) about 0.05% by weight; tonicity agent (glycerine or includes glycerine about 2.2% by weight; phosphate (combination of dibasic and monobasic) buffer (or other buffers such as Tris or sodium citrate buffer) of pH 6.0-8.0; sodium EDTA in the amount of about 0.02% or less by weight; an oil (e.g., castor oil) in the amount of about 1.25% by weight. Alternatively, the oil for the oil phase is a medium chain triglyceride in the range from 0.5-4%, typically at about 2%. To prepare this formulation, all water soluble components can be added and heated (about 60-70° C.) to make water the phase with buffer. A lipophilic solution is prepared using a lipophilic solvent (e.g., castor oil) and heating to about 60-70° C. Heterogeneous solution is formed by rapid addition of lipophilic solution into water phase followed by high shear mixing. The final solution is sterilized via 0.22 micron filter. Alternatively, sterilization can also be done by autoclaving at about 121° C. for 20 min. The sterilized heterogeneous solution is filled into single dose disposable tubes by BFS technology or the like.

Another general method for preparing a heterogeneous aqueous solution comprising a composition of the invention is as follows:
1. Mix Oil phase: Mix appropriate amounts of castor oil and polysorbate 80 until uniformity is obtained;
2. Mix Aqueous phase: Mix required amounts of Pemulen, water and glycerin until uniformity is obtained
3. Perform primary mixing of oil and aqueous phase mixtures from steps 1 and 2;
4. Perform high shear mixing and homogenization of mixture from step 3;
5. Confirm the ophthalmic solution properties via in process testing

Another example of a topical ophthalmic nanoemulsion formulation with its various components (w/w) useful for treating an eye disorder (e.g., dry eye syndrome) is as follows: it contains colloid particles with an average particle size of equal to or less than 0.2 μm and greater than 0.02 μm and has an oily core surrounded by an interfacial film. The size population distribution of the colloidal particles may be monomodal. The solution contains anywhere from 0.05% to 0.5% (e.g., 0.2%) alpha 2 adrenergic receptor agonist, cyclosporine from 0.01% to 0.3% (e.g., 0.075%), and 0.5 to 4% w/w (e.g., 1.25% w/w) castor oil or medium chain glycerides. It contains surfactants, preferably 0.5-4% by weight, polysorbate 80 (e.g. about 1.0% by weight); acrylate/C10-30 alkyl acrylate cross-polymer (about 0.05% by weight). Topical ophthalmic solution contains a tonicity agent or a demulcent component (e.g., glycerine, which can be in an amount of about 2.2% by weight), a buffer, such sodium citrate, tris-base to adjust the pH. The pH of this topical ophthalmic solution may be in the range of about 6.0 to about 8.0. The topical ophthalmic solution is therapeutically effective in increasing tear production.

Another example of a topical ophthalmic aqueous solution with its various components (w/w) useful for treating an eye disorder (e.g., dry eye syndrome) is as follows: it contains colloid particles with an average particle size of equal to or less than 0.2 μm and greater than 0.02 μm and has an oily core surrounded by an interfacial film. The size population distribution of the colloidal particles may be monomodal. The solution contains anywhere from 0.05% to 0.2% (e.g., 0.075%) alpha 2 adrenergic receptor agonist (e.g., brimonidine or a salt thereof) in weight to the total weight (w/w) of the oil phase, cyclosporine from 0.01% to 0.3% (e.g., 0.1%), and 0.5 to 4% w/w (e.g., 2% w/w) medium-chain triglycerides, 0.02% w/w benzalkonium chloride or no benzalkonium (preservative-free) for single dose sterile containers, and surfactants. The surfactants, for example, consist of a mixture of tyloxapol in an amount of 0.3% w/w and poloxamer in an amount of 0.1% w/w. The ophthalmic solution can include one or more oils selected from, castor, olive, soy, corn, mineral, cottonseed, safflower and sesame. The solution does not contain any significant amount (≤1%, typically ≤0.5%, often ≤0.1% and most often ≤0.01%) of substances capable of generating a negative charge and/or phospholipids. The ophthalmic heterogeneous solution can be used for treating a dry eye syndrome.

Yet another example of a topical ophthalmic nanoemulsion formulation with its various components (w/w) useful for treating an eye disorder is as follows: It contains brimonidine tartrate in an amount of about 0.2%; lifitegrast from 0.3% to 10% by weight, preferably 4% by weight, polysorbate 80 (e.g. about 1.0% by weight); acrylate/C10-30 alkyl acrylate cross-polymer (about 0.05% by weight); water q.s.; and castor oil in an amount of about 1.25% by weight. Topical ophthalmic solution contains a tonicity agent or a demulcent component (e.g., glycerine, which can be in an amount of about 2.2% by weight), a buffer, such sodium citrate, tris-base to adjust the pH. The pH of this topical ophthalmic solution may be in the range of about 6.0 to about 8.0. The topical ophthalmic solution is therapeutically effective in increasing tear production.

Yet another example of a topical ophthalmic aqueous solution with its various components (w/w) useful for treating an eye disorder is as follows: It contains brimonidine tartrate in an amount of about 0.02%; lifitegrast from 0.3% to 10% by weight, preferably 3% by weight, polysorbate 80 (e.g. about 1.0% by weight); acrylate/C10-30 alkyl acrylate cross-polymer (about 0.05% by weight); water q.s.; and castor oil in an amount of about 1.25% by weight. The alpha 2 adrenergic receptor agonist lifitegrast are the only active agents present in the topical ophthalmic solution but contains a tonicity agent or a demulcent component (e.g., glycerine, which can be in an amount of about 2.2% by weight), a buffer. The pH of this topical ophthalmic solution may be in the range of about 6.0 to about 8.0. The topical ophthalmic solution is therapeutically effective in increasing tear production.

Yet another example of a topical ophthalmic solution with its various components (w/w) is as follows: It contains brimonidine tartrate in an amount ranging from about 0.01% to about 0.5%, typically in an amount of about 0.2% by weight; cyclosporine from 0.01% to about 0.2%, typically in an amount of about 0.075% by weight; lifitegrast from 0.3% to 10% by weight, typically 4% by weight, carbomer homopolymer type B in an amount ranging from about 0.2 to about 0.6%, typically in an amount of about 0.4% or about 0.25%, and/or carbomer homopolymer type C in an amount ranging from about 0.4 to about 5% typically in an amount of about 4% or about 2.5%, and/or polycarbophil in an amount ranging from about 0.2% to about 0.5% typically in an amount of about 0.4% or about 0.2%; glycerin in an amount ranging from about 0.5% to about 1% typically in an amount of about 0.9%; benzalkonium chloride in an amount ranging from about 0.003% to about 0.01% typically in an amount of about 0.007%; edetate sodium in an amount ranging from about 0.03% to about 0.07% typically in an amount of about 0.05%; sodium chloride in an amount of up to about 0.09%, typically in an amount of about 0.06% or q.s. to isotonicity, or mannitol q.s. to isotonicity, or without isotonicity adjustors sodium chloride and mannitol; propylene glycol in an amount ranging from about 0.3% to about 0.6% typically in an amount of about 0.5%; water q.s., to 100 gms and sodium hydroxide or hydrochloric acid q.s., to adjust pH ranging from pH 6.0 to about 8.0. The topical ophthalmic solution is therapeutically effective for treating dry eye syndrome. Although preservatives such as benzalkonium chloride can be used in the formulations of the present invention as described in the non-limiting examples, typically the formulations are preservative-free.

Another example of a topical ophthalmic formulation with its various components (w/w) useful for treating an eye disorder (e.g., dry eye syndrome) is as follows: brimonidine tartrate in the amount of 0.02% to 0.3% by weight, typically about 0.1 to 0.2% and loteprednol etabonate 0.02 to 0.6% by weight, typically about 0.1 to 0.3%, surfactant such as Polysorbate 80 at about 0.02%-2% by weight or poloxamer/tyloxapol at about 0.1% and 0.25% by weight; carbomer copolymer (type A or type B) about 0.05% by weight; tonicity agent (glycerine or includes glycerine about 2.2% by weight; sodium citrate and tris buffer of pH 6.0-8.0, sodium EDTA in the amount of about 0.02% or less by weight; an oil (e.g., castor oil) in the amount of about 1.25% by weight. Alternatively, the oil for the oil phase is a medium chain triglyceride in the range from 0.5-4%, typically at about 2%. To prepare this formulation, all water soluble components can be added and heated (about 60-70° C.). Oil phase: the oil (e.g., castor oil) is heated to about 60-70° C. Coarse heterogeneous solution is formed by rapid addition of oil into water phase followed by high shear mixing. The final heterogeneous solution is sterilized via 0.22 micron filter. Alternatively, sterilization can also be done by autoclaving at about 121° C. for 20 min. The sterilized heterogeneous solution is filled into single dose disposable tubes by BFS technology or the like.

Yet another example of a topical ophthalmic combination formulation with its various components (w/w) useful for treating an eye disorder (e.g., dry eye syndrome) is as follows: brimonidine tartartrate in the amount of 0.02% to 0.3% by weight, preferably about 0.1 to 0.2% and loteprednol etabonate 0.02 to 0.6% by weight, preferably about 0.1 to 0.3%, povidone at about 0.6% by weight, poloxamer/tyloxapol at about 0.1% and 0.25% by weight; tonicity agent (glycerine or includes glycerine about 1 to 3% by weight; sodium citrate and tris buffer of pH 6.0-8.0; sodium EDTA in the amount of about 0.02% or less by weight.

### FORMULATION EXAMPLES

Ophthalmic pharmaceutical compositions can be formulated with the compositions shown in Table below. Heterogeneous solution formulation can be prepared according to the process described below where the water insoluble active(s) are added to the oil phase (e.g., castor oil) before introducing the oil phase to the aqueous phase.

Heterogeneous solution Formulation—Process Flow:
1. Oil phase: Mix appropriate amounts of castor oil and polysorbate 80 until uniformity is obtained;
2. Aqueous phase: Mix required amounts of Pemulen, water and glycerin until uniformity is obtained
3. Perform primary mixing of oil and aqueous phase mixtures from steps 1 and 2;
4. Perform high shear mixing and form heterogeneous solution from step 3;
5. Confirm heterogeneous solution properties via in process testing
   The above steps of the process flow need not be carried out in the same order.

| Ingredient | Range per mL | | Per mL | | Other substitutes | Function |
|---|---|---|---|---|---|---|
| Brimonidine | 0.2 to 5 | mg | 2 | mg | Tacrolimus, | Active Pharmaceutical |
| Tartrate | | | | | pimecrolimus | Ingredient* |
| Cyclosporin | 0.1 to 3 | mg | 1 | mg | voclosporin | |
| Lifitegrast | 3 to 100 | mg | 30 | mg | | |
| Loteprednol | 0.1 to 20 | mg | 2 | mg | | |
| etabonate | | | | | | |
| Castor Oil | 5-100 | mg | 12.5 | mg | Olive oil, Oleic | Oil Phase |
| | | | | | acid | |
| Polysorbate 80 | 0.1 to 40 | mg | 10 | mg | Polysorbate-20, | A component to both |
| | | | | | Poloxamer 188 | help facilitate the |
| | | | | | | formation of the |
| | | | | | | heterogeneous mixture |
| | | | | | | and improve its stability. |
| Pemulen TR-2 | 0.1 to 2 | mg | 0.5 | mg | N/A | Emulsion Stabilizer |
| Glycerin | 0-100 | mg | 22 | mg | Trehalose, sorbitol, | Tonicity-Adjusting |
| | | | | | mannitol, xylitol | Agent |
| Sodium Citrate | 0-20 | mg | 1.47 | mg | Phosphate, tris, | Maintain pH |
| Dihydrate | | | | | histidine, acetate, | |
| | | | | | succinate | |

| | | | | |
|---|---|---|---|---|
| Preservative | Optional | Optional | benzalkonium | For multidose and non- |
| | | | chloride (BAK), | sterile products |
| | | | stabilized | |
| | | | oxychloro complex | |
| | | | (Purite) | |
| Base for pH | | | pH 5 to 8 | Maintain pH |
| Acid for pH | | | pH 5 to 8 | Maintain pH |
| Water for | | QS | | Vehicle |
| Injection | | | | |

*For example, formulations containing different combinations of active ingredients are: bromonidine or brimonidine tartrate + cyclosporine; bromonidine or brimonidine tartrate + lifitegrast; cyclosporine + lifitegrast combination; bromonidine or brimonidine tartrate + cyclosporine + lifitegrast; brimonidine or brimonidine tartrate + loteprednol etabonate, etc.

| Ingredient | Range per mL | | Per mL | | Other substitutes | Function |
|---|---|---|---|---|---|---|
| Brimonidine | 0.2 to 5 | mg | 2 | mg | | Active Pharmaceutical |
| Tartrate | | | | | | Ingredient* |
| Cyclosporin | 0.1 to 3 | mg | 0.75 | mg | | |
| Castor Oil | 5-100 | mg | 12.5 | mg | Olive oil, Oleic | Oil Phase |
| | | | | | acid | |
| Polysorbate 80 | 0.1 to 40 | mg | 10 | mg | Polysorbate-20, | A component to both |
| | | | | | Poloxamer 188 | help facilitate the |
| | | | | | | formation of the |
| | | | | | | heterogeneous mixture |
| | | | | | | and improve its stability. |
| Pemulen TR-2/ | 0.1 to 2 | mg | 0.5 | mg | N/A | Emulsion Stabilizer |
| carbomer | | | | | | |
| copolymer type B | | | | | | |
| Glycerin | 0-100 | mg | 22 | mg | Trehalose, sorbitol, | Tonicity-Adjusting |
| | | | | | mannitol, xylitol | Agent |
| Sodium Citrate | 0-20 | mg | 1.5 | mg | Phosphate, tris, | Maintain pH |
| Dihydrate | | | | | histidine, acetate, | |
| | | | | | succinate | |

| | | | | |
|---|---|---|---|---|
| Preservative | Optional | Optional | benzalkonium | For multidose and non- |
| | | | chloride (BAK), | sterile products |
| | | | stabilized | |
| | | | oxychloro complex | |
| | | | (Purite) | |
| Base for pH, | | | pH 6 to 8 | Maintain pH |
| Tris-base | | | | |
| Acid for pH | | | pH 6 to 8 | Maintain pH |
| Water for | | QS | | Vehicle |
| Injection | | | | |

| Ingredient | Range per mL | | Per mL | | Other substitutes | Function |
|---|---|---|---|---|---|---|
| Brimonidine | 0.2 to 5 | mg | 2 | mg | | Active Pharmaceutical |
| Tartrate | | | | | | Ingredient* |
| Lifitigrast | 3 to 100 | mg | 40 | mg | | |
| Castor Oil | 5-100 | mg | 12.5 | mg | Olive oil, Oleic | Oil Phase |
| | | | | | acid | |
| Polysorbate 80 | 0.1 to 40 | mg | 10 | mg | Polysorbate-20, | A component to both |
| | | | | | Poloxamer 188 | help facilitate the |
| | | | | | | formation of the |
| | | | | | | heterogeneous mixture |
| | | | | | | and improve its stability. |
| Pemulen TR-2/ | 0.1 to 2 | mg | 0.5 | mg | N/A | Emulsion Stabilizer |
| carbomer | | | | | | |
| copolymer type B | | | | | | |
| Glycerin | 0-100 | mg | 22 | mg | Trehalose, sorbitol, | Tonicity-Adjusting |
| | | | | | mannitol, xylitol | Agent |
| Sodium Citrate | 0-20 | mg | 1.5 | mg | Phosphate, tris, | Maintain pH |
| Dihydrate | | | | | histidine, acetate, | |
| | | | | | succinate | |

| | | | | |
|---|---|---|---|---|
| Preservative | Optional | Optional | benzalkonium | For multidose and non- |
| | | | chloride (BAK), | sterile products |
| | | | stabilized | |
| | | | oxychloro complex | |
| | | | (Purite) | |
| Base for pH, | | | pH 6 to 8 | Maintain pH |
| Tris-base | | | | |
| Acid for pH | | | pH 6 to 8 | Maintain pH |
| Water for | | QS | | Vehicle |
| Injection | | | | |

The physical stability of these exemplary heterogeneous formulations can be monitored. For example, the heterogeneous solutions are allowed to stand for a period of time (e.g., 6 months) at 20 to 25° C., and the heterogeneity sizes are measured. The heterogeneity sizes within experimental error, should be identical at end of the test period to those measured right after the heterogeneous solution is prepared there by suggesting that there is no significant coalescence of the heterogeneity. Additionally, there should be no precipitation of the actives. Such results demonstrate that the heterogeneous formulations so prepared have superior physical stability.

Aqueous Formulation (Homogeneous and Heterogeneous Solutions):

General process for producing aqueous formulation of the compositions of the invention is provided below. Briefly, for X Volume (V) of final formulation—complete following steps:
1(a). Mix Carboxymethycellulose-Na in X/4 V of water
1(b). Mix Polysorbate, API (Brimonidine Tartrate and liftegrast) and stabilizer (Trehalose/Mannitol) in X/2 V of water
2. Mix mixtures from 1(a) and 1(b) together until formation of homogenous or heterogeneous solution.
3. Add 10× Stock of buffer (X/10 V) to final mixture from Step 2.
4. Adjust osmomolarity by adding NaCl Stocks to mixture from Step 3.
5. Adjust pH by adding HCl/NaOH to mixture from Step 4.
6. Make final volume to V by adding water to mixture from Step 5.
7. Perform filter sterilization.
8. Filling (BFS).

Combined Aqueous Formulations:

Example of combination product of aqueous formulation of Brimonidine Tartrate and Liftegrast:

| Aqueous | | |
|---|---|---|
| formulation | Range | Substitutes |
| Carboxymethyl- | 0.1 to 0.5% | Povidone, PEG 400, dextran, gelatin, |
| cellulose-Na | | hydroxy propyl methyl cellulose, |
| | | vinyl polymers |
| Trehalose | 1 to 12% | Glycerol, sucrose, mannitol |
| Polysorbate-80 | 0.01 to 4% | Tyloxapol, pluronic F-68, polaxomer |
| Citrate, Tris | 1 mM to | Phosphate, acetate, borate, histidine, |
| | 100 mM | succinate |
| Sodium Chloride | 0-140 mM | Magnesium chloride, calcium |
| | | chloride, potassium chloride etc |
| NaOH/HCL as per | N/A | N/A |
| requirement | | |
| pH | pH 5 to 8 | N/A |
| Osmolality | 200-400 | N/A |
| | mOSm | |

### Treatment Example

Several drops of a given formulation exemplified herein are administered to the eye(s) of a patient suffering from dry eye syndrome. Reduction of the symptoms becomes noticeable within a reasonable period. The treatment is repeated one or more times daily while the condition persists.

Combination Formulation:

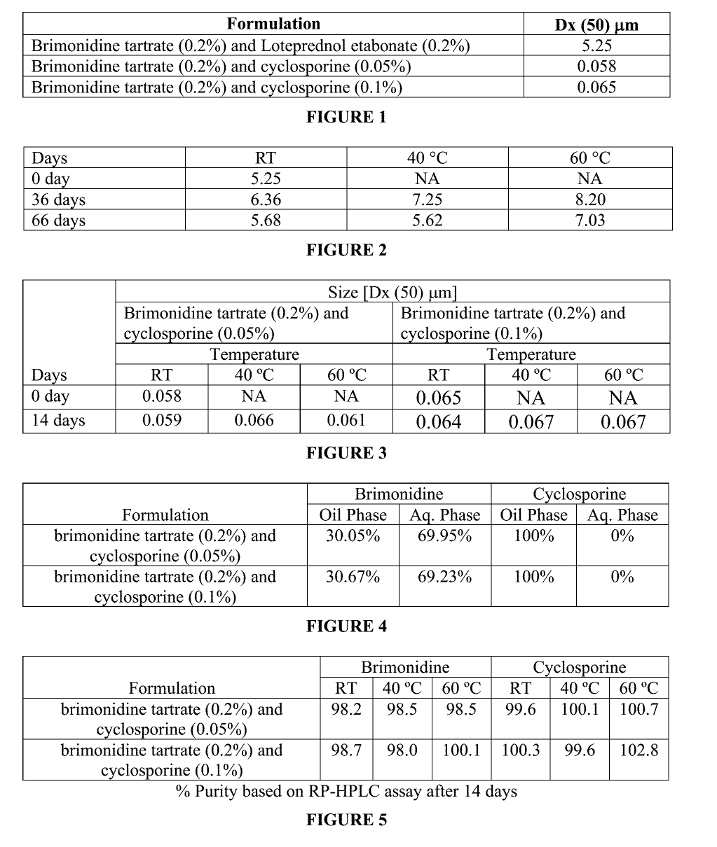

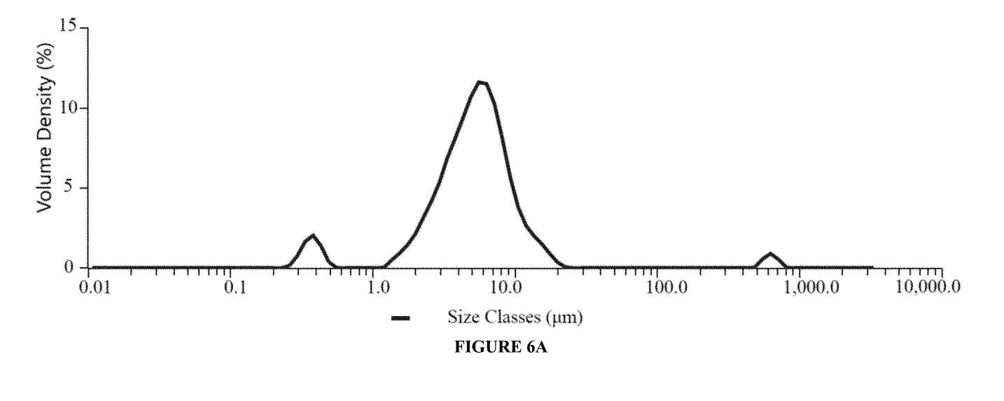

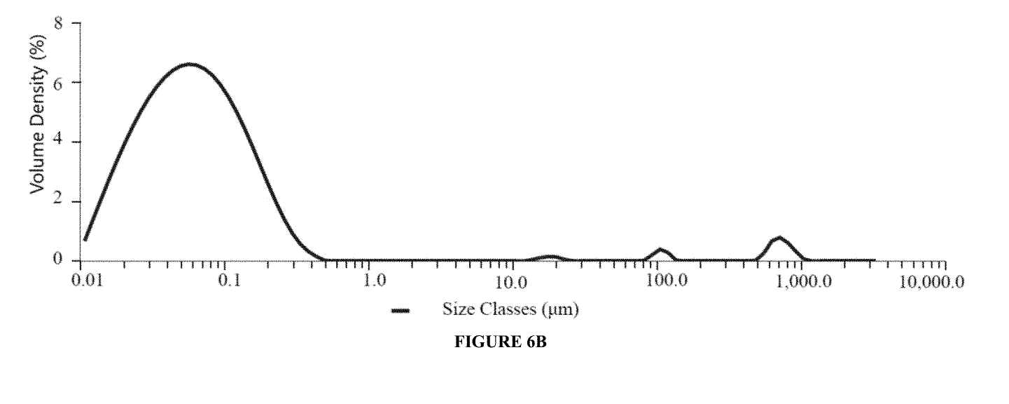

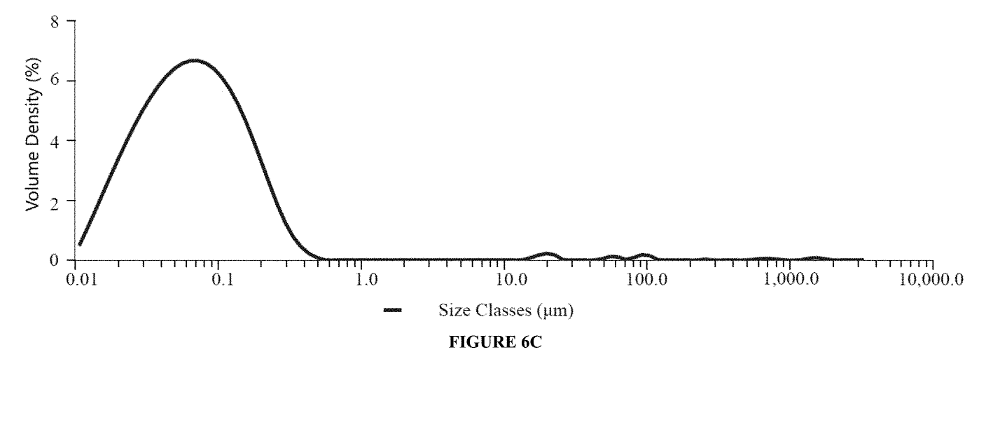

Various combinations of active pharmaceutical ingredient combinations were prepared using the processes disclosed herein. The particle or globule size distribution of these exemplary ophthalmic nanoemulsion formulations (e.g., homogeneous/heterogeneous solution; or suspensions) were determined using particle size analyzer, Mastersizer 3000™ (Malvern Panalytical, Malvern, UK). Formulations were dispersed in water during measurement and data were plotted as volume density (%) of particle v. size. Median size of globules/particles in formulation reported as Dx (50) as presented in tables in FIGS. 1-3. These data indicated that nanosized globule can be manufactured using process described in this application for nanoemulsion formulations (FIG. 3).

The physical stability nanoemulsion/solution/suspension formulations were also monitored using size distribution analysis over the extended time at various temperatures. For example, formulations are incubated for a period (e.g., 1-3 months) at storage temperature (e.g., 20° C. to 25° C.), and accelerated (e.g., 40° C.), and harsh (e.g., 60° C.) and the heterogeneity in sizes were measured. The heterogeneity in sizes, within experimental error, should be identical at end of the test period to those measured right after the nanoemulsion/suspension/solutions were prepared. Such consistency in the particle size would indicate that there is no significant coalescence of the heterogeneity at storage temperature. Even under accelerated stability temperature, there was no significant change in median size of globule/particles (FIGS. 2 and 3). These results demonstrate that the nanoemulsion/suspension formulations so prepared have superior physical stability.

The quantity of active pharmaceutical ingredients in various exemplary nanoemulsion formulations (e.g., homogeneous/heterogeneous solution; or suspensions) were determined using RP-HPLC methods. For combination formulation of brimonidine tartrate and cyclosporine, the HPLC method utilized a mixture of acetonitrile and a low pH buffer as an eluent. The chemical stability of APIs in nanoemulsion/solution/suspension formulations was determined using HPLC method after incubation for an extended time at various temperatures. For example, formulations are incubated at storage temperature (e.g., 20 to 25° C.), and accelerated (e.g., 40° C.), and harsh (e.g., 60° C.) and the purity was measured using HPLC method. The purity was substantially identical at the end of the 2 week test period compares to those measured right after the nanoemulsion formulation (FIG. 5) thereby suggesting that there is no significant degradation at storage temperature, accelerated and even at harsh temperature.

The partitioning of active pharmaceutical ingredients in oil and aqueous phases of these exemplary nanoemulsion formulations were determined using RP-HPLC methods. For combination formulation of brimonidine tartrate and cyclosporine, the HPLC method utilized a mixture of acetonitrile and a low pH buffer as eluent. In nanoemulsion formulation, oil and aqueous phase were separated using ultrafiltration unit by centrifugation process. Quantity of APIs in these oil and aqueous phase was quantified using HPLC method. Analyses showed that brimonidine partitioned in both phases of nanoemulsion formulation (˜30% in oil and ˜70% in aqueous phase) while cyclosporine partitioned substantially exclusively in oil phase. These results establish the unique differential characteristics of nanoemulsion formulation in partitioning of drug of different physicochemical characteristics in different phases of homogeneous or heterogeneous nanoemulsion formulations.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. An ophthalmic nanoemulsion formulation consisting of at least two active pharmaceutical ingredients selected from the group consisting of:
(a) an alpha 2 adrenergic receptor agonist;
(b) a beta-adrenergic receptor agonist;
(c) an immunosuppressant;
(d) a lymphocyte associated antigen antagonist;
(e) an anti-inflammatory;
(f) a beta-blocker;
(g) a prostaglandin analog;
(h) a histamine receptor antagonist;
(i) a carbonic anhydrase inhibitor; and
(j) an antibiotic.

2. The ophthalmic nanoemulsion formulation of claim 1, wherein a first active pharmaceutical ingredient is said alpha 2 adrenergic agonist, and a second active pharmaceutical ingredient is selected from the group consisting of: said immunosuppressant, said lymphocyte associated antigen agonist, said corticosteroid, said beta-blocker, said prostaglandins analog, said carbonic anhydrase inhibitor,

3. The ophthalmic nanoemulsion formulation of claim 2, wherein said immunosuppressant is selected from the group consisting of cyclosporine, tacrolimus, and a combination thereof.

4. The ophthalmic nanoemulsion formulation of claim 2, wherein said lymphocyte associated antigen agonist comprises Lifitegrast.

5. The ophthalmic nanoemulsion formulation of claim 2, wherein said corticosteroid is selected from the group consisting of prednisolone, methylprednisolone, difluprednate, prednisone acetate, prednisolone sodium phosphate, triamcinolone, fluocinolone; fluorometholone, betamethasone, medrysone, and a combination thereof.

6. The ophthalmic nanoemulsion formulation of claim 2, wherein said anti-inflammatory is selected from a group consisting of a corticosteroid, a non-steroidal anti-inflammatory drug (“NSAID”), thymosin beta 4, and a combination thereof.

7. The ophthalmic nanoemulsion formulation of claim 6, wherein said NSAID is selected from the group consisting of diclofenac, flubiprofen, ketorolac, ketorolac thromethamine, bromfenac, nepafenac, flurbiprofen, and a combination thereof.

8. The ophthalmic nanoemulsion formulation of claim 2, wherein said beta-adrenergic receptor agonist is selected from the group consisting of Dopexamine, Epinephrine, Isoprenaline, isoproterenol, levalbuterol, Salbutamol, albuterol, and a combination thereof.

9. The ophthalmic nanoemulsion formulation of claim 2, wherein said beta-blocker is selected from the group consisting of Timolol, Propranolo, Sotalol, nadolol, and a combination thereof.

10. The ophthalmic nanoemulsion formulation of claim 2, wherein said prostaglandins analog is selected from the group consisting of latanoprost, bimatoprost, travoprost, tafluprost, and a combination thereof.

11. The ophthalmic nanoemulsion formulation of claim 2, wherein said carbonic anhydrase inhibitor is selected from the group consisting of dorzolamide, methazolamide, brinzolamide, dichlorphenamide, and a combination thereof.

12. The ophthalmic nanoemulsion formulation of claim 1, wherein said active pharmaceutical ingredients consist of (i) brimonidine, a pharmaceutically acceptable salt thereof, or a combination thereof (ii) cyclosporine; and (iii) Lifitegrast or Loteprednol.

13. An aqueous ophthalmic solution comprising (i) brimonidine, a pharmaceutically acceptable salt thereof, or a combination thereof and (ii) cyclosporine.

14. The aqueous ophthalmic solution of claim 13 further comprising a pharmaceutically acceptable excipient.

15. The aqueous ophthalmic solution of claim 14, wherein said pharmaceutically acceptable excipient comprises:
(i) an emulsion stabilizing polymer;
(ii) a surfactant;
(iii) a tonicity modifier or a stabilizer selected from the group consisting of a polyol, a non-reducing disaccharide and a combination thereof; or
(iv) a combination thereof.

16. The aqueous ophthalmic solution of claim 13, wherein said solution is a nanoemulsion solution.

17. A method for treating an eye disorder, said method comprising administering to a subject in need of such a treatment a therapeutically effective amount of a composition of claim 1.

18. The method of claim 17, wherein said eye disorder is selected from the group consisting of: (i) a dry eye syndrome; (ii) ocular graft-versus-host-disease; (iii) ocular rosacea; (iv) allergic conjunctivitis; (v) autoimmune ocular surface disease; (vi) thygeson's superficial punctuate keratopathy; (vii) herpes zoster keratitis; (viii) Stevens-Johnson syndrome; (ix) keratitis; (x) conjunctivitis; (xi) blepharitis; (xii) blepharochalasis; (xiii) conjunctivochalasis; (xiv) blepharoconjunctivitis; (xv) blepharokeratoconjunctivitis; (xvi) post-operative inflammation or pain from ocular surgery; (xvii) scleritis; (xviii) episcleritis; (xix) anterior uveitis; (xx) iritis; (xxi) cyclitis; (xxii) ocular surface vascular disorder; (xxiii) ulcerative keratitis; (xxiv) photokeratitis; (xxv) dacryocystitis; (xxvi) eyelid disorder; (xxvii) congenital alacrima; (xxviii) xerophthalmia; (xxix) dacryoadenitis; (xxx) ocular hypertension; (xxxi) glaucoma; and (xxxii) ocular surface disorder induced by chemical burns, thermal burns, use of contact lenses, or physical insult to the ocular surface.

19. The method of claim 18, wherein said dry eye syndrome is selected from the group consisting of sjogren's syndrome, meibomian gland dysfunction and keratoconjunctivitis.

20. The method of claim 18, wherein said eyelid disorder comprises eyelid inflammation, pain or edema.
